(19) United States
(12) Patent Application Publication
(10) Pub. No.: US 2004/0127766 A1
(43) Pub. Date: Jul. 1, 2004

(54) LUBRICATING DILDO

(76) Inventor: Phil Chen, San Francisco, CA (US)

Correspondence Address: PHIL CHEN, 1535 LAKE ST., SAN FRANCISCO, CA, 94118, US

(21) Appl. No.: 10/328,982

(22) Filed: Dec. 26, 2002

(51) Int. Cl. A61F 5/00

(52) U.S. Cl. 600/38

(57) ABSTRACT

Lubricating Dildo with a hollow rigid approximately cylindrical housing. the housing encloses a pump, an oil holding reservoir, a motor, a power supply and tubing to transfer oil from the reservoir to apertures located at the front portion of the housing. A flexible sheath type covering encloses the housing. The sheath covering has a plurality of circumfrential pleats at its rear end and otherwise follows the contour of the housing. The pleats are flexible so that when the user presses on the rear end of the sheath, the pleats compress thereby allowing the the pump arm to be pressed inward thereby expelling oil from the front apertures. The motor is controlled by a combination switch and potentiometer located at the rear end of the sheath.

### CROSS REFERENCE TO RELATED APPLICATIONS

Not Applicable

### STATEMENT REGARDING FEDERALLY SPONSORED RESEARCH OR DEVELOPMENT

Not Applicable

### DESCRIPTION OF ATTACHED APPENDIX

Not Applicable

### BACKGROUND OF THE INVENTION

This invention relates generally to the field of sexual aids and more specifically to a lubricating dildo.

Devices for aiding in the pleasure of sexual experiences for both men and women have been in existance for many years.

One such device is known as a dildo. This device is usually a penis shaped member that can be inserted into the vagina of a woman, or in some cases, the anus of a man. Many different shapes and sizes of dildos are available in the market today. Some existing dildos include a vibrating means.

Although dildo's, including vibrating types, do provide pleasure to their user's, there is a deficiency in that the user must take time to lubricate the outside of the dildo for it to more easily slide into the intended orifice. Sometimes the dildo must be lubricated several times for it to provide maximum pleasure. This act of lubricating takes time and can take the spontaneity and pleasure out of the sexual act.

### BRIEF SUMMARY OF THE INVENTION

The primary object of the invention is to provide an improved dildo that allows the user to pump lubricating oil out of a plurality of apertures located on the outer surface of the dildo to increase the comfort and pleasure that said lubrication brings.

Another object of the invention is to provide an improved dildo that includes a vibrating means and method for controlling said vibrating means.

Other objects and advantages of the present invention will become apparent from the following descriptions, taken in connection with the accompanying drawings, wherein, by way of illustration and example, an embodiment of the present invention is disclosed.

In accordance with a preferred embodiment of the invention, there is disclosed a Lubricating Dildo comprising: a hollow rigid approximately cylindrical housing, said housing enclosing a pump, an oil holding reservoir, a motor, a power supply and tubing to transfer oil from said reservoir to apertures located at the front portion of said housing. A flexible sheath type covering encloses said housing, said sheath covering having a plurality of circumfrential pleats at its rear end and otherwise following the contour of said housing, said pleats being flexible so that when the user presses on the rear end of said sheath, said pleats compress thereby allowing the said pump arm to be pressed inward. Said motor controlled by a combination switch and potentiometer located at the rear end of the sheath. Said reservoir having a removable replaceable fill plug. Electrical wiring connects said potentiometer, motor and power supply.

### BRIEF DESCRIPTION OF THE DRAWINGS

The drawings constitute a part of this specification and include exemplary embodiments to the invention, which may be embodied in various forms. It is to be understood that in some instances various aspects of the invention may be shown exaggerated or enlarged to facilitate an understanding of the invention.

FIG. 1 is a side section view of the improved dildo of the present invention.

FIG. 2 is a perspective view of the improved dildo of the present invention.

### DETAILED DESCRIPTION OF THE PREFERRED EMBODIMENTS

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

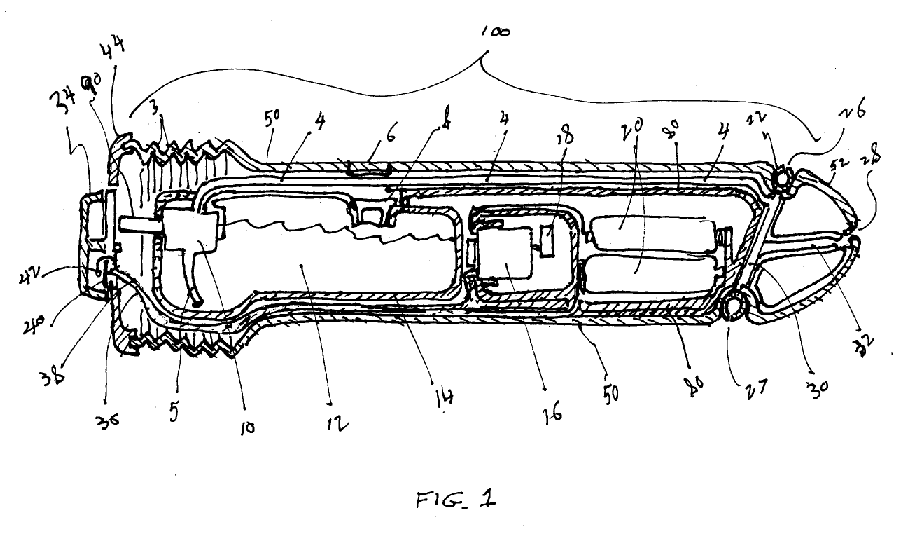

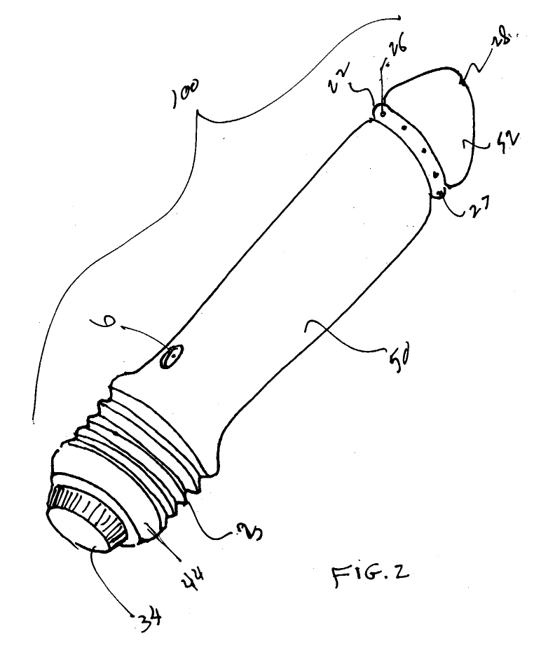

Referring now to FIG. 1 we see a side section view of the present invention 100. A rigid housing 80 made of injection molded plastic is generally cylindrical in shape and encloses a pump 10, an oil reservoir 14, a DC motor 16 and off center weight 18, a power supply 20 and tubing 4 that leads from the pump 10 to a tubular ring 22 and extension tube 32. An outer sheath 50 is made of flexible material such as elastomeric vinyl and follows the contour of the rigid housing 80. At the rear end of the housing 80 and sheath 50, the sheath 50 has a plurality of circumfrential pleats 3 so that when the user presses rear switch cover 34, the pleats 3 compress and allow pump arm 90 to be pressed in thereby pumping lubricating oil 12 trough tube 4 and to ring tube 22 where the oil can exit through a plurality of apertures 26, 27 and 28 thereby supplying lubricant to the outside of the sheath without the user having to interrupt her or his sexual activity to apply lubricant to the sheath 50. The motor 16 can be activated by the user turning the switch knob 34. A potentiometer 42, 40 built into the knob controls the amount of power sent by wires 36, 38 to motor 16 and power supply 20. The tubular ring 22 is recessed into sheath 50 so that it does not adversely affect the sliding contact with the user. Removable, replaceable plug 8 allows the user to add lubricating oil 12. The Back cover 44 of the sheath 50 is removable and replaceable so that the user can replace batteries 20 and remove plug 8. An optional additional plug 6 can be added to the sheath 50 so that the user does not have to remove sheath 50 to replace oil 12. FIG. 2 shows a perspective view of the present invention 100. Pleats 3 can be clearly seen. Apertures 26, 27, 28 can also be seen. Switch and potentiometer knob can be seen at the rear of the device 100. End cap 44 can be removed to reach the batteries 20 and fill plug 8.

The above description and illustrations show that the present novel invention 100 allows a user to easily add lubrication to the outside of the sheath whenever needed so that the use and convenience of the improved dildo of the present invention is significantly improved.

While the invention has been described in connection with a preferred embodiment, it is not intended to limit the scope of the invention to the particular form set forth, but on the contrary, it is intended to cover such alternatives, modifications, and equivalents as may be included within the spirit and scope of the invention as defined by the appended claims.

What is claimed is:

1. Lubricating Dildo comprising:
a hollow rigid approximately cylindrical housing;
said housing enclosing a pump, an oil holding reservoir, a motor, a power supply and tubing to transfer oil from said reservoir to apertures located at the front portion of said housing;
a flexible sheath type covering that encloses said housing;
said sheath covering having a plurality of circumfrential pleats at its rear end and otherwise following the contour of said housing;
said pleats being flexible so that when the user presses on the rear end of said sheath, said pleats compress thereby allowing the said pump arm to be pressed inward;
said motor controlled by a combination switch and potentiometer located at the rear end of the sheath;
said reservoir having a removable replaceable fill plug;
and electrical wiring connecting said potentiometer, motor and power supply.
